(19) United States
(12) Patent Application Publication
(10) Pub. No.: US 2006/0161088 A1
(43) Pub. Date: Jul. 20, 2006

(54) Continuous adhesive bandage roll for cutting into desired lengths

(76) Inventor: Thomas M. Voetsch, Kingston, NY (US)

Correspondence Address: JOHN MAIER, III, 660 Aaron Court, Kingston, NY, 12401, US

(21) Appl. No.: 11/035,430

(22) Filed: Jan. 14, 2005

(51) Int. Cl. A61F 15/00; A61F 13/00

(52) U.S. Cl. 602/43; 602/41; 602/42; 602/54

(57) ABSTRACT

An adhesive bandage formed in a continuous roll is provided for cutting into desired lengths depending upon the length of the wound. A base strip preferably of elastic material has a base adhesive layer on the inside surface of the base adhesive layer. A barrier layer, which is not as wide as the base layer is affixed to the base layer by the base adhesive layer. The barrier layer permits the passage of air and prevents the passage of fluids. A gauze adhesive layer is placed on the barrier layer and a gauze strip is placed on the barrier layer. The gauze strip is essentially of the same width as the barrier strip but the gauze strip is cut into gauze sections, preferably about fifteen millimeters being by cuts generally at right angles to the side edges of the gauze strip. Each gauze section is removable to expose the gauze adhesive layer on the barrier strip to permit securing the bandage to be secured about a wound.

### BACKGROUND OF THE INVENTION

1. Field of the Invention

This invention relates to adhesive bandages and, more specifically, to an adhesive bandage of continuous length that can be cut and applied to a wound of any reasonable length.

2. Prior Art

Cuts can occur on the body of a person that are long and not easily covered by adhesive bandages of what might be termed “standard size”. The cuts or wounds may not be so severe as to mandate wrapping or to require stitches installed by a physician. Such a wound can be caused by any number of situations. One such situation can occur when riding on an all terrain vehicle and passing a branch of a tree or shrub which may scratch an arm. With so-called “standard adhesive bandages” placing one such bandage next to the other to cover the wound, which can be a foot long, more or less, can require in excess of twelve bandages. Can be made in various widths and can cover even larger abrasions, and burns including a length of stitches following surgery.

An adhesive bandage that can be cut to order, even at the time of the injury, and immediately applied offers a great advantage. Obviously, a collection of bandages of varying lengths is not a good solution. A continuous adhesive bandage in a roll that can be cut anywhere from a “standard size” to the a longer required length does provide a desirable solution.

Various patents have been issued pertaining to bandages. The Adams, IV Patent, U.S. Pat. No. 3,835,992 provides an elongated strip carrier for a plurality of bandages but the bandages each individually produced are shown to be of generally the same size, and not as a continuous roll of adhesive bandage

The Lauritizen Patent, U.S. Pat. No. 4,530,353, provides for a bandage with a pad produced by a Z-fold but the generally continuous length of bandage is cut generally at right angles to the pad into bandages of one length apparently of standard size, and not of continuous length.

The Reed, et. al. Patent, U.S. Pat. No. 5,098,500, provides for a method to form a sheet-form of material including a porous sheet of absorbent, elastomeric segmented polyurethane. The bandage taught by Reed, et. al. is capable of being designed to suit particular wound types such as ulcers, donor sites, burns, as well as high exuding and low exuding wounds.

The Delmore, et. al., Pat. No. 5,939,339, teaches a bandage with a conformable, porous, self adhering elastomeric substrate that has a compressive force when extended along with an absorbent layer which is bonded to the self-adhering substrate.

The DeCandia, et. al. Patent, U.S. Pat. No. 6,174,399, teaches a method of making an elastic adhesive bandage where the bandage is initially produced in a continuous length. However, the continuos length is cut transversely to produce bandages of apparent standard size and all of which are of the same length.

### OBJECTS

The objects of this invention are to provide as follows:

1. An adhesive bandage that can be cut to any desired length at the time and place of an injury and applied to the wound. 2. An adhesive bandage that is convenient to both carry and use. 3. An adhesive bandage that permits removal of the absorbent part of the bandage at the ends to provide an adhesive surface about the entire perimeter of the absorbent pad. 4. An adhesive bandage that is economical and dependable.

These and other objects of the present invention will be apparent from the ensuing description and claims of the invention.

### SUMMARY

An adhesive bandage formed in a continuous roll is provided for cutting into desired lengths based upon the length of a wound. The adhesive bandage includes a base strip. An absorbent gauze strip is cut into gauze sections and each gauze section is substantially the same size. Means are provided for securing the gauze strip to the base strip. The base strip has marks on the outside surface to indicate the location of the gauze sections and each gauze section is removable, to expose and facilitate adhesive to skin.

### BRIEF DESCRIPTION OF THE DRAWINGS

FIG. 1 is a pictorial view of a typical dispenser of known existence partially broken away showing the roll of laminated bandage.

FIG. 2 is an exploded view of the bandage showing the layers and adhesive. Thickness of layers are exaggerated for clarity.

FIG. 3 is a pictorial view of the bandage with the layers together.

FIG. 4 is an exploded view similar to FIG. 2 but with the adhesive bandage turned over to show the adhesive base, fluid barrier and absorbent gauze layers.

FIG. 5 is a plan view of the bandage showing the gauze strip with gauze sections at the ends removed.

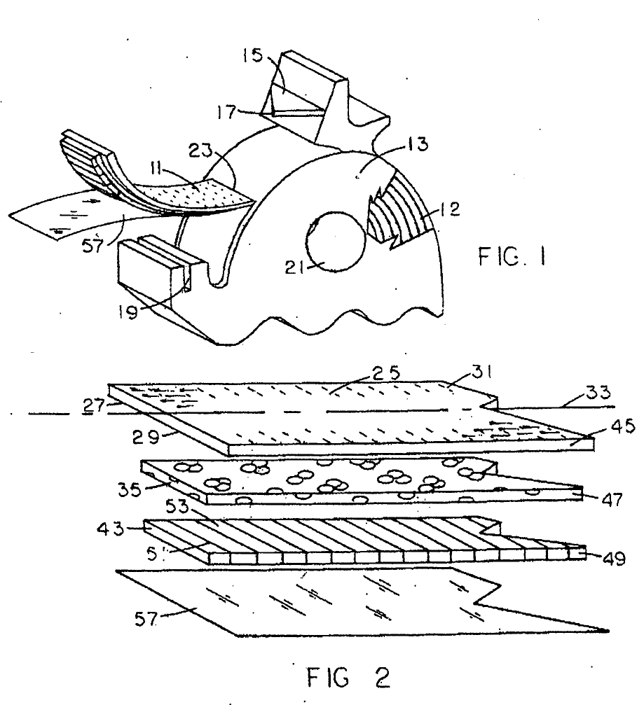

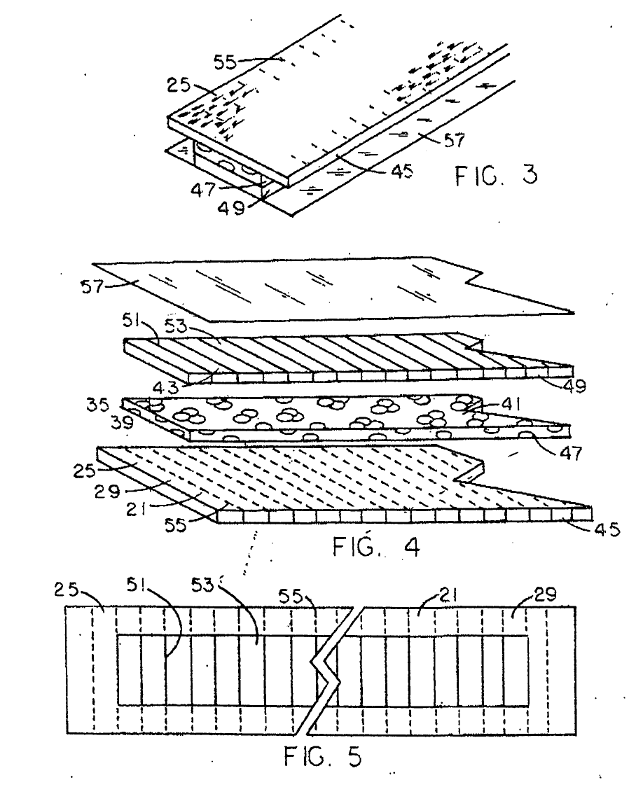

### DESCRIPTION OF THE NUMERALS

| NUMERAL | DESCRIPTION |
|---|---|
| 11 | ADHESIVE BANDAGE |
| 12 | ROLL OF ADHESIVE BANDAGE |
| 13 | DISPENSER |
| 15 | CUTTER |
| 17 | POINTED KNIFE OF CUTTER |
| 19 | CUTTER GROOVE |
| 21 | ROLL HOLDER |
| 23 | ADHESIVE BANDAGE OUTLET |
| 25 | BASE STRIP |
| 27 | BASE ADHESIVE LAYER ON INSIDE |
| | SURFACE (29) |
| 29 | INSIDE SURFACE |
| 31 | OUTSIDE SURFACE |
| 33 | CENTERLINE |
| 35 | FLUID BARRIER STRIP |
| 39 | FLUID BARRIER ADHESIVE LAYER |
| 41 | INSIDE SURFACE OF FLUID BARRIER (35) |
| 43 | GAUZE STRIP |
| 45 | SIDE EDGES (BASE) ADHESIVE LAYER (27) |
| 47 | SIDE EDGES (BARRIER) (35) |
| 49 | SIDE EDGES (GAUZE) (43) |
| 51 | CUTS ACROSS GAUZE STRIP |
| 53 | GAUZE SECTIONS |
| 55 | LINES ON FACE OF BASE STRIP (25) |
| 57 | COATED PROTECTIVE FILM |

### DETAILED DESCRIPTION OF THE PREFERRED EMBODIMENT

The adhesive bandage 11 is shown in FIG. 1 as a roll 12 within a dispenser 13. The dispenser 13 includes a cutter 15 which slides over the outside of the dispenser 13. The cutter 15 has a pointed knife 17 which pierces the adhesive bandage 11 and slides into a groove 19 thereby severing the adhesive bandage 11. Various other dispensing designs could be used. The dispenser 13 requires a roll holder 21 with an outlet 23 to feed the adhesive bandage 11 from the dispenser 13 to the cutter 15 for cutting the adhesive bandage 11 fed from the dispenser 13.

The adhesive bandage 11 has a base strip 25 which is elastic with base adhesive layer 27 on one side of the base strip 25 namely the inside surface 29 which faces the skin. The outside surface 31 is exposed when the adhesive bandage 11 is worn. The base strip 25 is formed from PVC foam tape or similar generally acceptable material. The inside surface 29 of the adhesive bandage 11 which has the base adhesive layer 27 is the side facing the skin.

Along the centerline 33 of the adhesive bandage 11 and affixed by the base adhesive layer 27 to the base strip 25 is a barrier strip 35 preferably of a thin film like material made from polyvinyl chloride or similar and which is perforated to allow for the passage of air to ventilate the wound while preventing the passage of fluid from the wound. The barrier strip 35 is located generally along the center line 33 of the base strip 25. The base strip 25 is wider than the barrier strip 35 and thus extends from the barrier strip 35 which provides for the base adhesive layer 27 to secure the perimeter of the adhesive bandage 11 to the skin.

A barrier adhesive layer 39 is applied to the outside surface 41 of the barrier strip which is the side of the barrier strip 35 remote from the base strip 25. A gauze strip 43, preferably having the same width as the barrier strip 35, is placed down on the outside surface 31 of the barrier strip 35 and is retained on the barrier strip 35 by the barrier adhesive layer 39 placed on the barrier strip 35 to hold the gauze strip 43. The barrier adhesive layer 39 may be formulated of a less aggressive adhesive so as to facilitate ease of removal of the gauze segments to therefore expose the adhesive to the skin. Further, a less aggressive adhesive permits the removal of the base strip 25 from the fluid barrier strip 35 and the gauze strip 43 assembly, should this assembly become adhered to the wound due to dried fluid. The gauze assembly can be soaked off with water. The base strip 25 has side edges 45 and likewise the barrier strip 35 has side edges 47 and the gauze strip 43 has side edges 49. These side edges 45, 47, 49 are generally parallel to one another. The gauze strip 43 has a series of cuts 51 across the gauze strip 43 situated generally at right angles to the side edges of the gauze strip 43. The cuts 51 create gauze sections 53 in the gauze strip 43 preferably about fifteen (15) millimeters in width, along the gauze strip 43. The cuts 51 extend from side edge 49 to side edge 49. Each gauze section 53 can be pulled from the barrier strip 35. By removing between one to three gauze sections 53 from the ends of the gauze strips 43 the barrier adhesive layer 39 on the barrier strip 35 is exposed sufficiently to secure the ends of the adhesive bandage 11 to the skin.

The base strip 25 has marking lines 55 on it formed from dye or heat which are preferably visible on both surfaces 29, 31 and must be visible on at least one of the surfaces 29, 31, preferably the outside surface 31 of the base strip 25. The lines 55 delineate the location of the gauze sections 53 and are specifically aligned with the series of cuts 51. The lines 55 on the outside surface 31 of the base strip 25, remote from the gauze strip 43 are of the greatest importance as these lines 55 permit the adhesive bandage 11 being cut from the roll 12 to be cut between two gauze sections 53.

Over the gauze strip 43, a coated paper or plastic protective film sheet 57, preferably having the width of the base strip 25, is placed down and adheres to the base strip 25. The protective sheet 57 can be readily removed from the base adhesive layer 27 on the base strip 25 but, until removed, preserves the cleanliness of the adhesive bandage 11.

To use the adhesive bandage 11, a length of adhesive bandage 11 is pulled from the roll 12 which is preferably located in a dispenser 13 and is pulled from the dispenser 13 to provide a length of adhesive bandage 11 that covers the wound being protected plus a reasonable additional length to extend beyond both ends of the wound. The adhesive bandage 11 is cut at that point. The protective sheet 57 is removed exposing the gauze layer. At least one and most likely three gauze sections 53 are removed from both ends and may be discarded. It is possible to fold the end gauze sections 53 over the remaining gauze sections 53 but this is probably not usually desirable. The adhesive bandage 11 is then placed over the wound and the base adhesive layer 27 is secured about the periphery of the adhesive bandage 11 to the skin by utilizing mostly the base adhesive layer 27 and also the barrier adhesive layer 39 where the gauze sections 53 have been removed. The less aggressive barrier adhesive layer 39 exposed at the ends will facilitate removal of the adhesive bandage 11 when removal is desired.

While a preferred embodiment is shown and described herein, it should be understood that the present disclosure is made by way of example only and that variations with regard to dimensions, i.e. various lengths and widths in the described device and its uses are possible within the scope of this disclosure without departing from the subject matter coming within the scope of the following claims, and a reasonable equivalency thereof, which claims are to be regarded as the invention.

1. An adhesive bandage in a continuous roll for cutting into desired lengths, such adhesive bandage comprising:
a base strip;
a gauze strip cut into gauze sections, each gauze section having substantially the same size, and
means for securing the gauze strip to the base strip, the base strip having marks to indicate the location of the gauze sections, each gauze section being removable.

2. The adhesive bandage according to claim 1 wherein the means for securing the gauze strip to the base strip includes a base adhesive layer on the base strip.

3. The adhesive bandage according to claim 1 wherein the means for securing the gauze strip to the base strip includes:
a base adhesive layer on the base strip.
a barrier strip affixed to the base adhesive layer; and
a gauze adhesive on the barrier strip, the gauze strip being affixed to the gauze adhesive layer.

4. The adhesive bandage according to claim 1 wherein:
the base strip has two side edges generally parallel to one another;
the gauze strip has two side edges generally parallel to one another and to the two side edges of the base strip, the gauze strip being cut generally at right angles to the side edges of the gauze strip.

5. An adhesive bandage in a continuous roll for cutting into desired lengths, such adhesive bandage comprising:
a base strip of elastic material;
a base adhesive layer on the base strip;
a barrier strip to permit the passage of air while prohibiting the passage of fluids, the barrier strip being affixed to the base adhesive layer on the base strip, the base strip wider than the barrier strip;
a gauze adhesive layer on the barrier strip;
a gauze strip affixed to the gauze adhesive layer on the barrier strip, the gauze strip being cut into gauze sections, each gauze section being removable from the barrier strip.

6. The adhesive bandage according to claim 5 wherein the material of the base strip is polyvinyl chloride, or similar suitable material.

7. The adhesive bandage according to claim 5 wherein:
the base strip has two side edges generally parallel to one another; and
the barrier strip has two side edges, the side edges of the barrier strip being substantially parallel to the side edges of the base strip.

8. The adhesive bandage according to claim 5 wherein:
the base strip has two side edges generally parallel to one another;
the barrier strip has two side edges, the side edges of the barrier strip being substantially parallel to the side edges of the base strip; and wherein
the gauze sections are approximately fifteen millimeters in width, the gauze strip being cut generally at right angles to the side edges of the gauze strip.

9. The adhesive bandage according to claim 5 wherein the base strip has an outside surface and an inside surface, the barrier strip being located on the inside surface, the outside surface of the base strip having lines to indicate the location of the gauze sections.

10. The adhesive bandage according to claim 5 wherein:
the base strip has two side edges generally parallel to one another;
the barrier strip having two side edges, the side edges of the barrier strip being substantially parallel to the side edges of the base strip; and wherein
the distance between the side edges of the barrier strip being substantially less than the distance between the side edges of the base strip, the barrier strip being generally located equi distant between the side edges of the base strip.

11. An adhesive bandage on a continuous roll for cutting into desired lengths, such bandage comprising:
a base strip having two side edges generally parallel to one another and having an inside surface and an outside surface;
a base adhesive layer on the inside surface of the base strip;
a barrier strip having two side edges to permit the passage of air and prohibit the passage of fluids, the barrier strip having an outside surface affixed to the base strip by the base adhesive layer and also having an inside surface, the side edges of the barrier strip being substantially parallel to one another and to the side edges of the base strip, the base strip extending from both side edges of the barrier strip;
a gauze adhesive layer on the inside surface of the barrier strip;
a gauze strip having two side edges affixed to the inside surface of the barrier strip by the adhesive on the barrier strip, the side edges of the gauze strip being parallel to one another and to the side edges of the base strip and the side edges of the barrier strip, the gauze strip being cut generally at right angles to the side edges of the gauze strip to form gauze sections of generally equal size, each gauze section being removable from the barrier strip, the base strip having marks on at least one side indicating the location of the gauze sections.

12. An adhesive bandage according to claim 11 further including a protective cover over the gauze strip.

13. The adhesive bandage according to claim 11 herein the material of the base strip is polyvinyl chloride or similar suitable material.

14. The adhesive bandage according to claim 11 wherein the gauze sections are approximately fifteen millimeters in width along the side edges of the gauze strip.

15. The adhesive bandage according to claim 11 herein the distance between the side edges of the barrier strip are substantially less than the distance between the side edges of the base strip.

16. The adhesive bandage according to claim 11 wherein the distance between the side edges of barrier strip are substantially less than the distance between the side edges of the base strip, the barrier strip being generally located equi distant between the side edges of the base strip, the gauze strip being generally the same size as the barrier strip and being aligned with the barrier strip.
